(19) United States
(12) Patent Application Publication
(10) Pub. No.: US 2018/0325184 A1
(43) Pub. Date: Nov. 15, 2018

(54) PATIENT SUPPORT

(71) Applicant: Dafydd Wyn Roberts, Conwy (GB)

(72) Inventor: Dafydd Wyn Roberts, Conwy (GB)

(21) Appl. No.: 15/773,848

(22) PCT Filed: Nov. 3, 2016
(86) PCT No.: PCT/GB2016/053408

§ 371 Date: May 4, 2018

(30) Foreign Application Priority Data
Nov. 10, 2015 (GB) 1519849.2

(51) Int. Cl. A41C 1/10; A41B 9/00; A61F 5/03

(52) U.S. Cl. CPC A41C 1/10; A61F 5/03; A41B 9/004

(57) ABSTRACT

A patient support (100), useful for the treatment of symphysis pubis dysfunction (SPD) and/or pelvic girdle pain (PGP), particularly in pregnant or postpartum women. The patient support (100) comprises a crotch section (110) adapted to bear against a patient's symphysis pubis in use. The crotch section (110) bearing against said patient's symphysis pubis may effectively relieve the discomfort and/or pain associated with SPD and/or PGP, particularly when the crotch section (110) comprises a rigid insert (700) for bearing against said patient's symphysis pubis. The use of the patient support (100) in the treatment of pelvic girdle pain and/or symphysis pubis dysfunction is also described. A rigid insert portion (700) adapted for attachment to a patient support (100) and adapted to bear against a patient's symphysis pubis in use is also described.

### FIELD

The present invention relates to a patient support, in particular to a patient support for use in the treatment of pelvic girdle pain and/or symphysis pubis dysfunction. In particular the invention relates to a patient support for use by pregnant women suffering from pelvic girdle pain and/or symphysis pubis dysfunction.

### BACKGROUND

The symphysis pubis is the mid-line cartilaginous joint uniting the superior rami of the left and right pubic bones. Symphysis pubis dysfunction (SPD) is a condition characterised by excessive movement of the symphysis pubis, either anterior or lateral movement. Such movement and the resultant misalignment of the pelvis can cause pain, often severe pain. SPD is most commonly associated with pregnancy and childbirth and may affect up to one in four pregnant women to varying degrees, with around 7% of sufferers continuing to experience serious symptoms postpartum.

During pregnancy, the hormone relaxin is released slowly over the course of the pregnancy to relax the muscles in the pelvic region so that the pelvis can expand sufficiently during childbirth. In SPD, relaxin may be released more rapidly than normal, having the effect that the muscles in the pelvic region are relaxed too quickly for the associated ligaments to adjust. This results in the pelvis being misaligned causing the ligaments to stretch excessively, which is believed to cause or at least contribute to the pain associated with SPD.

SPD is associated with pelvic girdle pain (PGP) and the names are often used interchangeably.

The main symptom of SPD is usually pain or discomfort in the pelvic region, often centred on the joint at the front of the pelvis (the symphysis pubis). Sufferers also frequently experience pain in the lower back, hips, groin, lower abdomen, and legs. The severity of the pain can range from mild discomfort to extreme and prolonged suffering. SPD can be associated with or cause depression on account of the physical discomfort endured by sufferers. Sufferers of SPD may find it difficult and painful to carry out everyday activities such as walking or climbing stairs.

SPD may ultimately lead to disabilities or physical damage if not properly treated. Current treatments involve using crutches to assist walking and taking pain relief medication, neither of which address the underlying cause of SPD or provide an effective relief from the discomfort and pain.

### SUMMARY OF THE INVENTION

It is one aim of the present invention, amongst others, to provide a patient support that addresses at least one disadvantage of the prior art, whether identified here or elsewhere, or to provide an alternative to existing patient supports. For instance it is an aim of the invention to provide a patient support which is useful for the treatment of SPD and/or PGP.

According to aspects of the present invention, there is provided a patient support as set forth in the appended claims. Other features of the invention will be apparent from the dependent claims, and the description which follows.

Throughout this specification, the term “comprising” or “comprises” means including the component(s) specified but not to the exclusion of the presence of other components.

The term “consisting of” or “consists of” means including the components specified but excluding addition of other components.

Whenever appropriate, depending upon the context, the use of the term “comprises” or “comprising” may also be taken to encompass or include the meaning “consists essentially of” or “consisting essentially of”, and may also be taken to include the meaning “consists of” or “consisting of”.

The optional features set out herein may be used either individually or in combination with each other where appropriate and particularly in the combinations as set out in the accompanying claims. The optional features for each aspect or exemplary embodiment of the invention as set out herein are also to be read as applicable to any other aspect or exemplary embodiments of the invention, where appropriate. In other words, the skilled person reading this specification should consider the optional features for each exemplary embodiment of the invention as interchangeable and combinable between different exemplary embodiments.

According to a first aspect of the present invention there is provided a patient support comprising a crotch section adapted to be positioned over and apply inward pressure to a patient's symphysis pubis.

The patient support is suitably a wearable garment for providing support and/or inward pressure to parts of a patient's (a wearer's) body.

Suitably the crotch section covers at least the area of said patient's skin adjacent to said patient's symphysis pubis. Optionally clothing may be arranged in use between the crotch section and said patient's skin. Suitably the patient support is intended to be worn by a patient over underwear and/or beneath outer clothing such as dresses, trousers, shirts etc.

In use the patient support provides inward pressure to said patient's symphysis pubis through the crotch section. Said inward pressure may be alternatively or additionally defined as upward support or an upward lifting (when said patient is standing or sitting upright).

Suitably in use the crotch section is biased against and/or bears upon said patient's symphysis pubis.

Suitably the patient support is worn by said patient in a manner which forces the crotch section against the symphysis pubis towards said patient's abdomen.

By inward pressure we do not mean to refer to merely producing a reaction force from a patient's body which may be produced on wearing clothing such as underwear or trousers.

Suitably said inward pressure is sufficient to relieve the discomfort and/or pain experienced by sufferers of SPD and/or PGP. Said inward pressure may achieve the discomfort and/or pain relief by supporting the symphysis pubis joint and/or re-aligning the symphysis pubis into its normal state (i.e. before onset of SPD/PGP) and/or bringing together the opposing sides of the symphysis pubis joint.

The symphysis pubis may also be known as the pubic symphysis.

The inventors have found that the patient support of this first aspect when fitted to a patient to provide said inward pressure on the symphysis pubis may effectively relieve the discomfort and/or pain associated with SPD and/or PGP. The patient support has been found to be more effective that known patient supports which do not produce said inward pressure on the symphysis pubis.

The patient support may produce said inward pressure in use by being tightly fitted to said patient's body and/or restrained on an anchor point or points on said patient's body. For example a waist, hip, shoulder or neck of said patient may provide an anchor point for fitting the patient support onto in order to generate the required bias and/or inward pressure on said patient's symphysis pubis through the crotch section.

Suitably the patient is a women, suitably a pregnant women or a women who has recently given birth, for example a postpartum women.

Women and in particular pregnant and postpartum women are more often affected by SPD and/or PGP than other population groups and have been shown to benefit from wearing the patient support of this first aspect.

Suitably the crotch section comprises a rigid portion adapted to sit against and apply said inward pressure to said patient's symphysis pubis.

By rigid we mean the rigid portion substantially maintains its shape in use when applying inward pressure to said patient's symphysis pubis.

The inventors have found that a rigid portion arranged in the crotch section of the patient support may improve the effectiveness of the discomfort and/or pain relief provided by the inward pressure on the patient's symphysis pubis. The rigid portion may facilitate the achievement of sufficient inward pressure.

The rigid portion may be formed as an integral part of the patient support. Alternatively the rigid portion may be detachable. For example the crotch section may comprise a sleeve or slot for receiving and retaining the rigid portion in use. The rigid portion may be provided with flaps adapted to fold over and embrace the crotch section to retain the rigid portion on the crotch section.

The rigid portion may be formed from a polymeric material, for example to provide a lightweight rigid portion. The rigid portion may be hollow, for example to provide a lightweight rigid portion.

Suitably the rigid portion has a curved upper surface adapted to sit against and apply said inward pressure to said patient's symphysis pubis.

By upper surface we mean the surface of the rigid portion which is intended to be held against said patient's symphysis pubis by the patient support, in use.

The inventors have found that such a curved surface improves the effectiveness of the discomfort and/or pain relief provided by the inward pressure on the patient's symphysis pubis. Such a curved surface may also improve the wearing comfort experienced by the patient.

Suitably the curved upper surface is curved in a longitudinal direction with respect to the crotch section, namely in a direction passing between said patient's legs. Suitably the curved upper surface is curved in a lateral direction with respect to the crotch section, namely in a direction passing through each of said patient's legs. Suitably the curved upper surface is curved in both a longitudinal and a lateral direction with respect to the crotch section.

Suitably the curved upper surface is ergonomically shaped to compliment said patient's symphysis pubis.

Suitably the curved upper surface is teardrop shaped.

By teardrop shaped we mean a three dimensional oval shape with one narrower end and one wider end, for example an ovoid shape. Suitably the narrower end of the teardrop shape in intended to face away from the front of said patient, in use.

Suitably the rigid portion comprises a substantially flat lower surface for facilitating the fitting of the rigid portion into the crotch section of the patient support. Suitably the rigid portion comprises a lower surface which complements a shape which the crotch section adopts in use, in the region of said patient's symphysis pubis, for facilitating the fitting of the rigid portion into the crotch section of the patient support, for example a curved shape in the longitudinal direction.

Suitably the rigid portion comprises a curved, suitably teardrop shaped upper surface and a substantially flat lower surface.

Suitably the rigid portion comprises a curved, suitably teardrop shaped upper surface and a lower surface having a curved shape in the longitudinal direction.

The inventors have found that providing a rigid portion with a curved and/or teardrop shaped upper surface may provide more effective discomfort and/or pain relief provided by the inward pressure on the patient's symphysis pubis and may also improve the wearing comfort experienced by the patient.

Suitably the patient support of this first aspect comprises a shoulder strap adapted to restrain and bias the crotch section against said patient's symphysis pubis.

Suitably the patient support comprises a pair of shoulder straps, one for placing over each shoulder of said patient in use.

The inventors have found that shoulder straps may provide a convenient anchor for the patient support to perform its function of applying inward pressure to the patient's symphysis pubis. The shoulder straps may allow such inward pressure to be applied whilst maintaining the patient's comfort and also may allow easy fitting of the patient support.

Suitably the patient support comprises a releasable attachment adapted to connect the shoulder strap to the crotch section.

By releasable attachment we mean that at least one end of the shoulder straps can be detached from the patient support in order to facilitate placing the support on said patient, and can be re-attached to the patient support in use to restrain and bias the crotch section against said patient's symphysis pubis.

Suitably the shoulder strap or straps are attached to the crotch section, suitably directly attached, suitably using a hook and loop fastening. The patient support may be provided with a fastening loop through which the shoulder strap or straps are passed to form a loop from the patient support and around the patient's shoulder. The shoulder strap may then be folded back over itself and attached to itself, for example using a hook and loop fastening. This arrangement may allow for the easy adjustment of the patient support, for example to accommodate different sized patients and/or to vary the inward pressure applied to the patient's symphysis pubis in order to optimise the discomfort/pain relief.

Suitably the patient support of this first aspect comprises a hip section adapted to wrap circumferentially around said patient's hips and compress said hips towards each other.

By wrap circumferentially around we mean in the manner of a waist belt but lower down on a patient's body than a belt would typically be worn.

Compressing said hips towards each other may be additionally or alternatively defined as applying an inward pressure in the direction of said patient's sacroiliac joints.

The inventors have found that applying such compression/inward pressure may provide an additional benefit of discomfort and/or pain relief in some patients, possibly due to such patients suffering from a pain or dysfunction of one or both sacroiliac joints. Said compression/inward pressure may achieve the discomfort and/or pain relief by supporting the sacroiliac joints and/or re-aligning the sacroiliac joints into their normal state (i.e. before onset of SPD/PGP) and/or bringing together the opposing sides of the sacroiliac joints.

Suitably the hip section comprises a releasable attachment to facilitate fitting the hip section of the patient support onto said patient.

Suitably the hip section is attached to the crotch section and/or the shoulder strap or straps if present, suitably attached through a back section. The hip section may comprise two ends adapted to allow releasable attachment of the two ends to each other, for example the ends may be provided with hook and loop fasteners. Such arrangements may allow for the easy adjustment of the hip section of the patient support, for example to accommodate different sized patients and/or to vary the inward pressure applied to the patient's hips in order to optimise the discomfort/pain relief.

Suitably the hip section has a width of at least 5 cm, suitably at least 7 cm, suitably at least 9 cm.

Suitably the hip section has a width sufficient to cover substantially all of a patient's hip areas and thereby apply more effective compression of the hips towards each other.

The inventors have found that better hip compression over a larger area of the hips than achieved by known patient supports may provide better discomfort/pain relief in patients, particularly patients suffering from discomfort/pain centred in the sacroiliac joints.

Suitably the patient support of this first aspect comprises a waist section adapted to wrap around said patient's waist and provide upward support for said patient's abdomen.

The waist section is typically higher up on said patient's body than the hip section, if present. Suitably the waist section fits under and provides support for a pregnant women's abdomen, i.e. where the developing baby is carried by the pregnant women.

By support we mean an upward lifting force to counteract the weight of the patient's abdomen.

The inventors have found an additional benefit to the patient may be provided by the patient support comprising a waist section by relieving the additional discomfort or pain associated with the weight of the abdomen, in particular in pregnant women.

The waist section may be adapted to be tight fitting around said patient's waist in order to provide said upward support for said patient's abdomen. Alternatively, the waist section may be attached to a shoulder strap in order to provide an anchor point for said upward support/upward lifting force for said patient's abdomen.

Suitably the waist section is attached to the crotch section and/or the shoulder strap or straps if present, suitably attached through a back section. The waist section may comprise two ends adapted to allow releasable attachment of the two ends to each other, for example the ends may be provided with hook and loop fasteners. Such arrangements may allow for the easy adjustment of the waist section of the patient support, for example to accommodate different sized patients and/or to vary the upward support for said patient's abdomen in order to optimise the discomfort/pain relief.

Suitably the patient support comprises a hip section and a waist section, linked through a back section.

Suitably the hip section and waist section are formed such that the hip section and waist section merge, or are linked by a panel of material, at a rear side of the patient support (intended to sit against said patient's back in use) to form a back section.

The hip section and the waist section being linked in this manner allows the support to be formed from a single piece of material, which may provide a simplicity of construction and use, and also may allow the patient support to provide more effective support to the patient's lower back area and provide a further benefit in relieving discomfort/pain centred in this area.

Suitably the patient support comprises a lumbar section adapted to provide support to said patient's lower back.

Suitably the lumbar section is provided as a separate piece of material releasably attachable to a rear side of the patient support (intended to sit against said patient's back in use), suitably to a back section of the patient support, for example using hook and loop fasteners.

Alternatively the lumbar section may be permanently attached to the patient support.

Suitably the lumbar section is adjustable to accommodate different sized patients and/or to vary the support provided to the patient's lower back in order to optimise the discomfort/pain relief.

The lumbar section may allow the patient to be given additional support to the lower back area in order to provide additional support for the patient's lower back and therefore an additional benefit in discomfort and pain relief.

The lumbar section may be formed from the same type of material as the rest of the patient support.

Suitably the crotch section and any shoulder strap, hip section, waist section or back section, if present, are permanently attached to the patient support on at least one end thereof.

Suitably the patient support is formed from a single piece of material.

Suitably the crotch section and any shoulder strap, hip section, waist section or back section, if present, are permanently attached to and formed from a single piece of material.

The patient support being constructed from a single piece of material may provide a simplicity of construction and use.

Suitably the patient support is constructed from a material, suitably comprising a fabric, with a high resistance to elastic deformation. In other words the material is suitably adapted to apply the required inward pressure on said patient's symphysis pubis by transferring a tensioning force applied to the patient support, for example on tightly fitting the patient support to said patient, to the crotch section. A support constructed from a material with a relatively low resistance to elastic deformation, for example known patient supports formed from relatively highly elastic material, would be unable to transfer such a tensioning force to a crotch section to apply sufficient pressure on the patient's symphysis pubis. However, suitably the patient support is constructed from a material, suitably a fabric, which is slightly elastic, for example to allow for said patient's movement whilst wearing the patient support.

Suitably the patient support is constructed from a breathable material, suitably a breathable fabric, suitably with a high resistance to elastic deformation.

Suitably the patient support is constructed from a material which is comfortable to wear against said patient's skin.

Suitably the patient support is constructed from a material comprising a breathable rubber. Suitably the patient support is constructed from a breathable rubber, suitably a breathable coated rubber, suitably a breathable rubber coated with a fabric comprising lycra and/or nylon, suitably a breathable rubber coated with a fabric comprising lycra, suitably a breathable rubber coated with a fabric comprising lycra and nylon.

Suitably the patient support is constructed from a material with an unbroken loop finish or outer surface. Suitably the patient support is constructed from a material, suitably a rubber, suitably a breathable rubber, coated with a fabric having an unbroken loop finish or outer surface. Suitably the patient support is constructed from a material coated with a fabric comprising lycra, the fabric having an unbroken loop finish or outer surface.

By unbroken loop finish we mean the surface comprises loop structures which can serve as the loop in a hook and loop fastening arrangement.

The patient support being constructed from a material with an unbroken loop finish or outer surface has the advantage that a separate piece of material does not need to be attached to the patient support during construction in order to provide the “loop” part of any hook and loop fastening arrangement present. In addition, anywhere on the surface of the patient support may provide the “loop” part of any hook and loop fastening arrangement, allowing greater scope for the adjustment or optimisation of the fit and function of the patient support than would be possible with a defined “loop” part, for example a patch of “loop” material sewn onto the patient support.

In embodiments of the patient support comprising a hip section and a waist section, the patient support suitably comprises a gap between the hip section and the waist section, suitably where said patient's hip would be, in use. Suitably such a gap facilitates the movement of the legs of the patient, for example when walking or in the act of sitting down. Suitably the gap allows the patient's skin in said region of the hip to move unencumbered by the patient support when walking or in the act of sitting down.

Suitably the crotch section and/or the shoulder strap and/or the hip section and/or the waist section and/or the lumbar section are adjustable to accommodate different patients and/or to optimise the discomfort/pain relief provided by the patient support.

Suitable arrangements to provide the required adjustment are discussed above and other suitable arrangements may be known in the art.

According to a second aspect of the present invention, there is provided a patient support according to the first aspect for use in the treatment of pelvic girdle pain and/or symphysis pubis dysfunction.

Suitably the patient support is for use in the treatment of women with pelvic girdle pain and/or symphysis pubis dysfunction, suitably pregnant women, suitably postpartum women.

In trials on patients who are pregnant women suffering from SPD and/or PGP, the patient support of the present invention has been shown to provide effective discomfort and pain relief when worn by the patient.

According to a third aspect of the present invention there is provided a rigid insert portion adapted to enable placement in a patient support and to enable the application of inward pressure to a patient's symphysis pubis.

The suitable features and advantages of the rigid insert portion of this third aspect are as described in relation to the rigid portion of the first aspect.

The patient support which the rigid insert portion is adapted to enable placement in may be a patient support according to the first aspect.

According to a fourth aspect of the present invention, there is provided a patient support comprising:

a crotch section adapted to be positioned over and apply inward pressure to a patient's symphysis pubis;
a shoulder strap adapted to restrain and bias the crotch section against said patient's symphysis pubis; and
a hip section adapted to wrap circumferentially around said patient's hips and compress said hips towards each other.

According to a fifth aspect of the present invention, there is provided a patient support comprising:

a crotch section adapted to be positioned over and apply inward pressure to a patient's symphysis pubis;
a shoulder strap adapted to restrain and bias the crotch section against said patient's symphysis pubis;
a hip section adapted to wrap circumferentially around said patient's hips and compress said hips towards each other; and
a waist section adapted to wrap around said patient's waist and provide upward support for said patient's abdomen.

According to a sixth aspect of the present invention, there is provided a patient support comprising:

a crotch section adapted to be positioned over and apply inward pressure to a patient's symphysis pubis;
a shoulder strap adapted to restrain and bias the crotch section against said patient's symphysis pubis;
a hip section adapted to wrap circumferentially around said patient's hips and compress said hips towards each other; and
a waist section adapted to wrap around said patient's waist and provide upward support for said patient's abdomen;
wherein the crotch section comprises a rigid portion adapted to sit against and apply said inward pressure to said patient's symphysis pubis.

According to the present invention there is provided a patient support substantially as described herein, and/or a patient support substantially as described herein with reference to the accompanying Figures and/or a patient support substantially as shown in the accompanying Figures.

### BRIEF DESCRIPTION OF THE DRAWINGS

For a better understanding of the invention, and to show how example embodiments may be carried into effect, reference will now be made to the accompanying drawings in which:

FIG. 1 is a perspective view of a patient support (100) according to the first aspect of the present invention fitted onto a patient (200).

FIG. 2 is a diagram of a patient's (200) pelvic bones.

FIG. 3 is a perspective view of the patient support (100) of FIG. 1 in a first stage of fitting to a patient (200).

FIG. 4 is a perspective view of the patient support (100) of FIG. 1 in a second stage of fitting to a patient (200).

FIG. 5 is a perspective view of the patient support (100) of FIG. 1 in a third stage of fitting to a patient (200).

FIG. 6 is a perspective view showing the rear of the patient support (100) of FIG. 1.

FIG. 7 is a perspective view of a rigid portion (700) according to the third aspect of the present invention.

### DETAILED DESCRIPTION OF THE EXAMPLE EMBODIMENTS

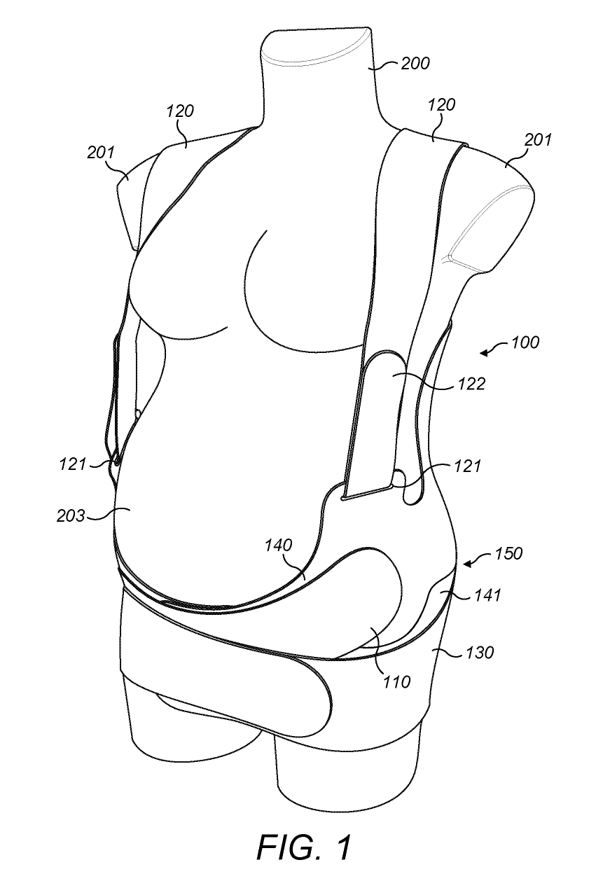

FIG. 1 shows a patient support (100) comprising a crotch section (110), shoulder straps (120), hip section (130), waist section (140) and back section (150). The patient support (100) may also comprises a lumber section (160, not shown in this view). The patient support (100) is mainly constructed from a breathable rubber coated with a fabric comprising lycra and nylon, the fabric having an unbroken loop finish. This construction makes the patient support (100) slightly elastic to allow the patient support (100) to provide the required inward pressure, through the crotch section (110), to the patient's symphysis pubis. The slightly elastic nature of the patient support (100) also improves its wearing comfort. The breathable rubber of the patient support (100) also functions to wick moisture away from the patient, again to improve the wearing comfort of the patient. Each of the crotch section (110), shoulder straps (120), hip section (130) and waist section (140) are releasably attached using a “hook” fastening section which can co-operate with the unbroken loop finish on the fabric coating of the patient support (100). This facilitates a patient putting on the patient support and adjusting the patient support to suit their particular body shape and size and optimise each of the inward pressures exerted by the crotch section on the patient's symphysis pubis, the hip compression exerted by the hip section (130) and the upward support exerted by the waist section (140).

The crotch section (110) is approximately 8 cm wide, the shoulder straps (120) are approximately 6 cm wide, the hip section (130) is approximately 9 cm wide and the waist section (140) is approximately 10 cm wide. The width in each case referring to the dimension across the outer surface of each part in a direction perpendicular to the loop which each part forms when the patient support (100) is fitted to a patient (200), in other words in a lateral direction. These dimensions may be varied to provide a patient support which fits a particular size of patient.

FIG. 1 shows the patient support (100) fully fitted onto a mannequin (200) representing a pregnant women patient. Crotch section (110) is partially obscured from view by the hip section (130). Shoulder straps (120) are attached to waist section (140) via a retaining loops (121) arranged on waist section (140) through which each of the shoulder straps (120) pass to form a closed loop shoulder strap between each of the mannequin's (patient's) shoulders (201) and the waist section (140) of the patient support (100). Each shoulder strap (120) is attached to itself using a hook fastening section arranged at end (122). Crotch section (110) is attached by hook fastening section onto the waist section (140) adjacent to where the shoulder straps (120) are linked to the waist section (140) through the retaining loops. The attachment of crotch section (110) to waist section (140) allows the required inward pressure to be applied to the patient's symphysis pubis in order to provide pain relief for a sufferer of SPD or PGP, for example. Hip section (130) is attached to the patient support (100) via back section (150) and fastens to itself via a hook fastening section at the front of the patient (200) in order to provide the required compression force on the hips of the patient (200) to provide the additional benefits of discomfort/pain relief and hip realignment to the sufferer of SDP or PGP. The waist section (140) is arranged underneath and in contact with the patient's abdomen (203) in order to lift and thus support the weight of the patient's developing baby, for example. Gap (141) provided between the hip section (130) and waist section (140) facilitates the movement of the legs of the patient (200) when fitted with the patient support (100), for example when walking or in the act of sitting down.

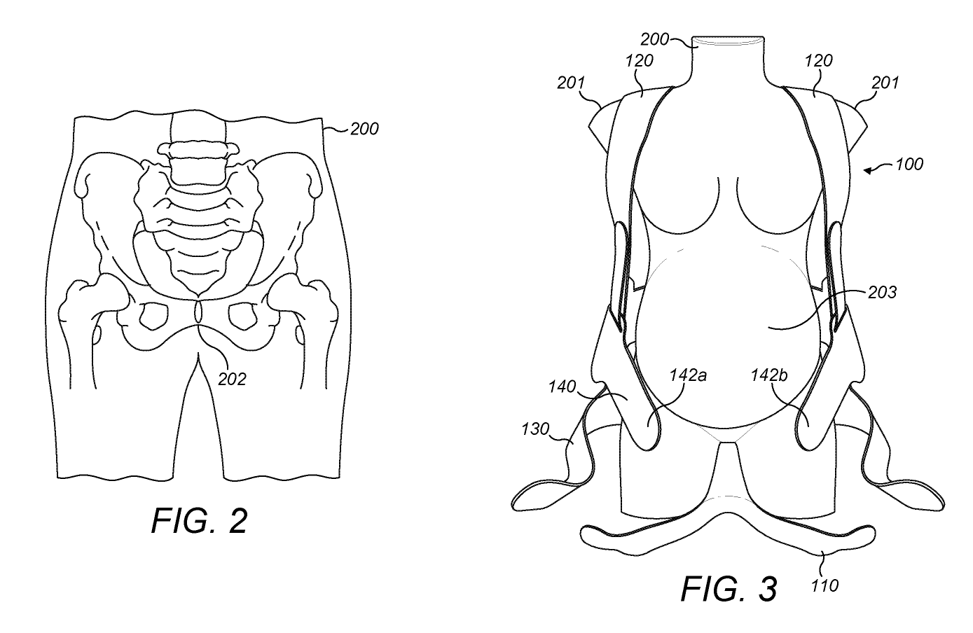

FIG. 2 shows a diagram of the patient's (200) pelvic bones and also shows the location of the symphysis pubis joint (202).

FIGS. 3, 4 and 5 show different stages of fitting the patient support (100) to a patient (200). In FIG. 3 the patient support (100) has been arranged on a patient (200) with the shoulder straps (120) placed on the shoulders (201) of patient (200) with the back section (150, not shown) arranged to contact the lower back of the patient (200). The crotch section (110) is passed between the legs of patient (200) and in contact with the patient's skin adjacent to the symphysis pubis joint, or in contact with any underwear in contact with the patient's skin adjacent to the symphysis pubis joint. The waist section (140) comprises ends (142a and 142b) of which end (142b) comprises a hook fastening section to allow attachment to end (142a) in order to wrap waist section (140) circumferentially around the patient (200) underneath and in contact with the patient's abdomen (203). This may provide relief from the additional discomfort or pain associated with the weight of the abdomen by applying upward support/lifting to the patient's abdomen.

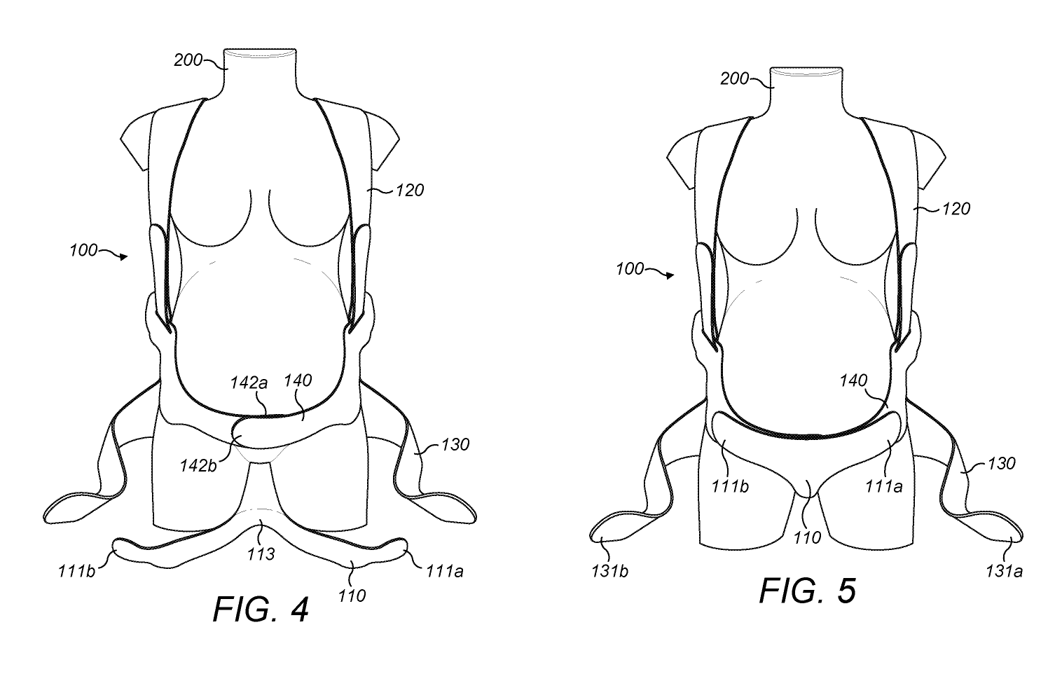

FIG. 4 shows the patient support (100) wherein the ends (142a and 142b) of the waist section (140) have been attached to each other as described above. Crotch section (110) comprises a gusset (113) and ends (111a and 111b) both of which comprise a hook fasteneing section to allow the crotch section (110) to fasten to waist section (140) adjacent to the shoulder straps (120) attached to the waist section (140). Fastening the crotch section (110) to the waist section (140) tightly in this manner provides the required inward pressure on the symphysis pubis through the tensioning achieved by using the shoulder straps (120) and may relieve the discomfort and pain associated with SPD/PGP. A teardrop shaped rigid portion (700, see FIG. 7) may be attached to the crotch section (110) at the gusset (113) in order to increase the inward pressure applied to the symphysis pubis joint by the crotch section (110) in the patient support (100), in use.

FIG. 5 shows a patient support (100) wherein the crotch section (110) has been attached to waist section (140) using the hook fastening sections arranged on the waist section (140) and on ends (111a and 111b) of the crotch section (110). The hip section (130) comprises a hook fastening section arranged on end (131b) in order to allow the hip section (130) to be attached to itself at end (131a) when wrapped circumferentially around the hips of patient (200) to produce the inward pressure on the patient's hips which may provide the additional discomfort/pain relief discussed above. As mentioned above, the fully assembled patient support (100) fitted to patient (200) is shown in FIG. 1.

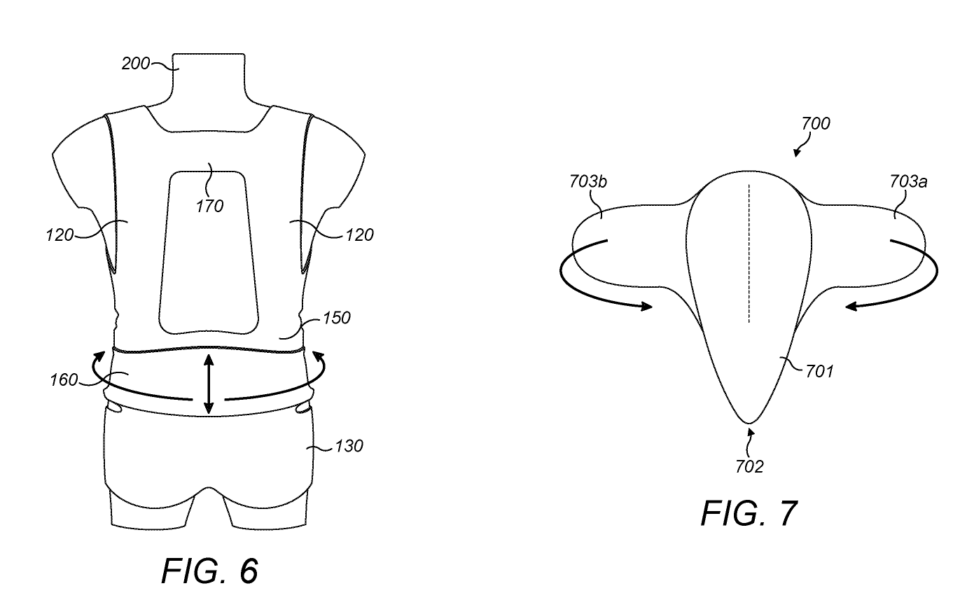

FIG. 6 shows a rear view of the patient support (100) fitted onto a patient (200). FIG. 6 shows that the hip section (130) and the waist section (140) are joined at the rear of the patient support (100), forming a back section (150) which covers substantially the whole of the buttocks and lower back of the patient (200). This arrangement of back section (150), waist section (140) and hip section (130) provides a simplicity of construction and an improved hip and/or abdomen support compared to known patient supports. FIG. 6 also shows the back strap (170) which links the shoulder straps (120) together to hold the shoulder straps (120) in the correct position for comfortable and effective wearing of the patient support (100). FIG. 6 also shows an optional lumber section (160) which may be attached to the back section (150) of the patient support (100) in order to provide support to the lower back of the patient (200) (as shown by the arrows) and thereby provide additional discomfort and/or pain relief to the patient (200).

FIG. 7 shows a teardrop shaped rigid portion (700) comprising a main rigid part (701) for bearing against and applying inward pressure to a patient's symphysis pubis joint in use, for example when fitted to a patient support such as the patient support (100) worn by the patient (200). The rigid portion (700) comprises flaps (703a and 703b) for folding over and embracing (as shown by the arrows) a crotch section such as (110) of a patient support such as (100) to retain the rigid portion (700) in place on the crotch section (110). The retaining of the rigid portion (700) may be facilitated by a hook fastening section attached to the rigid portion (700), for example on the flaps (703a and 703b), co-operating with an unbroken loop finish on the lycra coating of the patient support (100) or by using a suitable adhesive, for example. Tip (702) is intended to be arranged in a crotch section (110) of a patient support (100) pointing away from the front of the patient (200).

The patient support (100) has been tested on pregnant women patients suffering to varying degrees with SPD and/or PGP. The support (100) was found to be effective in relieving the discomfort and/or pain experienced by the pregnant women before they were fitted with the patient support (100). The relief of pain and discomfort was found to be most effective when the teardrop shape rigid portion (700) was attached to crotch section (110), as described above, and therefore worn against the symphysis pubis joint by the patient. The patients who have tested the patient support (100) have found that the often debilitating discomfort or pain caused by SPD and/or PGP they were suffering from was almost completely relieved by the patient support (100), allowing the patients to complete everyday tasks such as walking or climbing stairs with minimal difficulty or discomfort.

The example embodiments described above may therefore address the hitherto unmet medical need for relief from the pain or discomfort associated with SPD and/or PGP, allowing sufferers, for example pregnant women, to remain normally active during an occurrence of SPD and/or PGP, for example throughout a pregnancy and/or a postpartum period.

In summary, a patient support, useful for the treatment of symphysis pubis dysfunction (SPD) and/or pelvic girdle pain (PGP), particularly in pregnant or postpartum women is disclosed. The patient support comprises a crotch section adapted to bear against a patient's symphysis pubis in use. The crotch section bearing against said patient's symphysis pubis may effectively relieve the discomfort and/or pain associated with SPD and/or PGP, particularly when the crotch section comprises a rigid insert for bearing against said patient's symphysis pubis. The use of the patient support in the treatment of pelvic girdle pain and/or symphysis pubis dysfunction is also disclosed. A rigid insert portion adapted for attachment to a patient support and adapted to bear against a patient's symphysis pubis in use is also disclosed.

Although a few preferred embodiments have been shown and described, it will be appreciated by those skilled in the art that various changes and modifications might be made without departing from the scope of the invention, as defined in the appended claims.

Attention is directed to all papers and documents which are filed concurrently with or previous to this specification in connection with this application and which are open to public inspection with this specification, and the contents of all such papers and documents are incorporated herein by reference.

All of the features disclosed in this specification (including any accompanying claims, and drawings), and/or all of the steps of any method or process so disclosed, may be combined in any combination, except combinations where at least some of such features and/or steps are mutually exclusive.

Each feature disclosed in this specification (including any accompanying claims, and drawings) may be replaced by alternative features serving the same, equivalent or similar purpose, unless expressly stated otherwise. Thus, unless expressly stated otherwise, each feature disclosed is one example only of a generic series of equivalent or similar features.

The invention is not restricted to the details of the foregoing embodiment(s). The invention extends to any novel one, or any novel combination, of the features disclosed in this specification (including any accompanying claims, and drawings), or to any novel one, or any novel combination, of the steps of any method or process so disclosed.

1. A patient support comprising a crotch section adapted to be positioned over and apply inward pressure to a patient's symphysis pubis.

2. The patient support according to claim 1, wherein the crotch section comprises a rigid portion adapted to sit against and apply said inward pressure to said patient's symphysis pubis.

3. The patient support according to claim 2, wherein the rigid portion has a curved upper surface adapted to sit against and apply said inward pressure to said patient's symphysis pubis.

4. The patient support according to claim 3, wherein the curved upper surface is ergonomically shaped to compliment said patient's symphysis pubis, suitably the curved upper surface is teardrop shaped.

5. The patient support according to any preceding claim, comprising a shoulder strap adapted to restrain and bias the crotch section against said patient's symphysis pubis.

6. The patient support according to claim 5, comprising a releasable attachment adapted to connect the shoulder strap to the crotch section.

7. The patient support according to any preceding claim, comprising a hip section adapted to wrap circumferentially around said patient's hips and compress said hips towards each other.

8. The patient support according to claim 1, comprising:
a shoulder strap adapted to restrain and bias the crotch section against said patient's symphysis pubis; and
a hip section adapted to wrap circumferentially around said patient's hips and compress said hips towards each other.

9. The patient support according to claim 7 or claim 8, wherein the hip section has a width of at least 5 cm.

10. The patient support according to any preceding claim, comprising a waist section adapted to wrap around said patient's waist and provide upward support for said patient's abdomen.

11. The patient support according to claim 10, comprising a hip section according to any one of claim 7, 8 or 9, wherein the waist section is linked to the hip section via a back section.

12. The patient support according to any preceding claim, comprising a lumbar section adapted to provide support to said patient's lower back.

13. The patient support according to any preceding claim formed from a single piece of material.

14. The patient support according to any preceding claim constructed from a material comprising a breathable rubber.

15. The patient support according to any preceding claim, wherein the crotch section and/or the shoulder strap and/or the hip section and/or the waist section and/or the lumbar section are adjustable to accommodate different patients and/or to optimise discomfort and/or pain relief provided by the patient support.

16. The patient support according to any preceding claim for use in the treatment of pelvic girdle pain and/or symphysis pubis dysfunction.

17. A rigid insert portion adapted to enable placement in a patient support and to enable the application of inward pressure to a patient's symphysis pubis.

18. A patient support substantially as described herein, and/or a patient support substantially as described herein with reference to the accompanying Figures and/or a patient support substantially as shown in the accompanying Figures.
